(19) United States
(12) Patent Application Publication
(10) Pub. No.: US 2009/0143850 A1
(43) Pub. Date: Jun. 4, 2009

(54) ENDOPROSTHESIS AND DELIVERY SYSTEM FOR ITS PLACEMENT WITHIN A PATIENTS VESSEL AND USES FOR THIS ENDOPROSTHESIS AND DELIVERY SYSTEM

(76) Inventor: Pierre Galvagni Silveira, Florianopolis-SC (BR)

Correspondence Address: KNOBBE MARTENS OLSON & BEAR LLP, 2040 MAIN STREET, FOURTEENTH FLOOR, IRVINE, CA, 92614, US

(21) Appl. No.: 12/198,742

(22) Filed: Aug. 26, 2008

(30) Foreign Application Priority Data
Nov. 30, 2007 (BR) PI 0704464-0

(51) Int. Cl. A61F 2/06; A61F 2/84

(52) U.S. Cl. 623/1.11; 623/1.35

(57) ABSTRACT

The present invention relates to an endoprosthesis composed of (a) two complementary bodies, body “A” and (b) a body “B”, with the said bodies being formed by proximal, intermediate and distal regions and comprising a self-expanding structure of resistant and flexible material and a graft wherein the said bodies “A” and “B” have a bifurcation for use where a main vessel bifurcates into at least two secondary branches, such as in the case of the treatment of aneurysms in the abdominal aorta.

The invention further includes a delivery system for the endoprosthesis of the present invention, as well as its use in the treatment of aneurysms.

### CROSS-REFERENCES AND RELATED APPLICATIONS

This application claims priority to Brazilian patent application No. PI 0704464-0 filed in Brazil on Nov. 30, 2007, the disclosure of which is expressly incorporated herein by reference in its entirety for any purpose.

### FIELD OF THE INVENTION

The present invention relates overall to a device and a method for repairing the arterial system at the point where a principal artery bifurcates into at least two secondary arteries. More specifically, the invention is intended for the repair of an aneurysm in the vicinity of the aorta. One of the embodiments of the invention relates to a modular intravascular device termed a self-expanding percutaneous endoprosthesis used in the treatment of aneurysms, more specifically of the abdominal aorta. The said device is composed of two complementary bodies, namely, body “A” and body “B”, which both comprise self-expanding metal structures (a set of several stents) and a graft of appropriate tissue. A second embodiment of the invention includes the use of the delivery system of the invention for implanting an endoprosthesis in a patient needing such treatment. Yet a third embodiment provides a delivery system for placing the self-expanding percutaneous endoprosthesis device at its intended position.

### BACKGROUND

An aneurysm of the abdominal aorta is a dilatation of the walls of this vessel in the abdominal region. The aorta is the bodies' main artery, ascending from the the heart's left ventricle to arch around and descend through the thorax and abdomen to finally divide into the two common iliac arteries that supply blood to the pelvis and lower limbs. Aneurysms usually occur in the abdominal part of the aorta below the kidneys. Failure to treat this condition may eventually result in the rupture of the dilatation (aneurysm) causing a massive haemorrhage in a very short period of time with fatal consequences. This is the reasons that treatments such as implanting a reinforcing prosthesis inside the dilated part of the aorta walls are vital to save patients lives. Despite abdominal aortic aneurysms being the most common, they are not restricted to the abdominal area. Aneurysms may also occur, for example, in the aorta thoracica.

The disruptions caused by of the abdominal aortic aneurysms are very serious and may lead to death. Until recently, treatment of aortic aneurysms consisted of invasive surgery methods for inserting a graft inside the aorta to reinforce the artery. Such a procedure requires a surgical incision to allow access to the vessel, which may result in rupture of the aneurysm due to the sudden reduction in the external pressure exerted by the neighboring organs and tissues which are displaced during acess procedure. Quite apart from this serious issue, other risk factors include loss of blood and consequent weakness, anuria and low blood pressure associated to the abdominal aortic aneurysm. As a result of the inherent risks and complexity of surgical procedures, several alternative devices and methods have been proposed for implanting a graft inside vessels for the treatment of aneurysms.

However, despite the advances represented by the use of stent and stent-graft devices, they have revealed failings both with regard their implanting processes and performance. As described in US Pat. 2006/184229, these failings may be classified into four main categories. Type I failings are related to the occurrence of leakage between the vascular endoprosthesis and the vessel walls in the area of the proximal aorta immediately above the aneurysm and, therefore, results in continued blood flow to the aneurysmal sac, which thus maintains the pressure at this point and favours continued expansion and consequent rupture of the aneurysm. Type I failings may also be caused by the irregular shape of the vessel and/or calcified topography of the aorta lumen which results in poorly inserted circular prostheses in non-circular lumens of the aorta. Type II failings are related to blood flowing through collateral vessels in the dilated area of the aneurysm which requires a further embolisation procedure. Type III failings are of mechanical origin and result from excessive wear of the metal/non-metal interface or the poor integrity of a connection or connections between the modular components of a prosthesis. Lastly, the type IV failings are related to excessive porosity of the prosthesis walls which allows the blood to migrate through the walls despite the soundness of all mechanical seals and connections. To remedy usual Type I failings, US Pat. 2006/184229 proposes an implantable prosthesis with a radialy expandible tubular body with at least one flap extending through it.

The U.S. Pat. No. 5,562,724 describes an endovascular graft prosthesis to be positioned in—or close to—a bifurcation of the arterial system of a patient, with this prosthesis comprising a main tubular body having a bag-shape and provided with two outlet openings wherein the said main body is intended for location in the principal upstream artery above the bifurcation and having tubular legs joining the main body and adapted to extend into the two downstram arteries. The positioning of the main body in relation to the radially expansive stent devices and the entire assembly in the arteries is done through the use of guide wires. Although such a device does represent an advance compared to former techniques involving surgical procedures, it still presents failings related to its unfavourable displacement along the blood flow and the precision required when correctly positioning the device at the time of implant. The device described in US Pat. 2007/027531 also uses a system of guide wires to facilitate the impant operation of the device, which comprises at least one filamentous tubular member having a distal extremity and a proximal extremity with a hollow nucleus to receive the guide wire that helps position the device at the intended location.

Document U.S. Pat. No. 6,802,859 proposes a bifurcated graft implant having a trunk portion and a portion with independent flexible legs wherein the entire assembly (main portion+legs) may constitute a unitary body or be formed of modular elements. To ensure the flexibility of the bifurcation, this graft is supported by a stent lattice throughout. Despite this device being self-expanding and having appropriate flexibility at the region of bifurcation, it remains difficult to implant at the intended location.

Another device for the repair of abdominal aortic aneurysms is proposed in U.S. Pat. No. 6,942,691. This device comprises a modular graft that includes two elements configured to be inserted into each other over an extension sufficiently long as to form a resistant seal yet remaining flexible enough to adapt to the region of the bifurcation. The said device comprises a first and second modular element with each expanding from an originally compressed state so as to allow implantation at the intended aneurysm location. The graft described in U.S. Pat. No. 6,942,691 is practical since it allows insertion of both component elements at an intended location but, nevertheless, it presents inconveniences related to the stability of its placement and the relative safety of the large blood flow expected through it.

A solution to facilitate the implant of a device in the region of na aneurysm of the abdominal aorta is proposed in document US 2003/120338. This solution relates to providing means to allow the use of a catheter having a very small diameter in the delivery systems for devices within the bodies of patients. The proposed device includes a graft having proximal and distal extremities and is provided with a connector member arranged or fixed at one or both extremities, having one or more connector elements wherein the said connector member may be enclosed within multiple layers of the graft body section. Despite this solution being of interest due to the use of a reduced diameter catheter, this device described in US 2003/120338 is complex and presents the disadvantage of being difficult to position correctly at its intended location. A similar device also presenting the same disadvantage is described in document US 2006/173533 (corresponding to EP 1464301).

To correct the issues of stent graft instability, unwanted displacement from the required position and material fatigue, document WO 2001/67993 proposes a stent graft assembly comprising a main body having an ipsilateral leg and a contralateral stump that, combined, form a bifurcation at the distal extremity. A delivery system for this stent graft assembly is also proposed.

Document WO 2006/014952 (corresponding to US 2006/025850) describes an endoprosthesis comprising (i) a main body having a tubular structure configured to attach firmly to a vessel and serve as a seal preventing blood from reaching the aneurysm, (ii) a section constituted of two legs allowing the passage of fluids to the main body and having multiple stent elements and, (iii) a graft attached to the main body and the two legs. This type of endoprosthesis presents the disadvantage of greater implanting difficulty since it consists of a unitary body insufficiently flexible to adjust well at its intended position.

Another fundamental aspect for the treatment of aneurysms using endoprosthses relates to the methods of implanting these and appropriate means for this delicate operation.

An endoprosthesis delivery system for the adequate implant of stents or graft stents should incorporate the following desirable features: (a) A minimum diameter should be maintained during insertion to avoid trauma and/or unneccessary difficulties during the implanting operation: (b) Means to facilitate the precise visualisation and positioning of the delivery catheter to ensure correct positioning of the device: (c) Reliable and reproducible expansion of the metal structure and graft metal structure allowing complete expansion to operational diameter with no deviations to the degree or efficiency of expansion: (d) Reliable and reproducible uncoupling or release of the metal structure and the graft metal structure from the catheter body; (e) Efficient retreival or removal of the delivery catheter without disturbing the recently implanted stent or graft stent: (f) Ease of verifying the occurrence of biological fluid (i.e. blood) leakage around the endoprosthesis (endo-leakage) following delivery and implantation within the vessel.

Several delivery systems have been proposed aiming to ensure these features. For example, document U.S. Pat. No. 6,379,372 (corresponding to PI 9712034) describes a delivery and implant system for use inside a body lumen (e.g. a blood vessel) for a radially expandable endoluminal prosthesis with the said system comprising: (a) a delivery catheter, (b) an introducer assembly, and (c) a dilator. Despite this system allowing the safe introduction of an expandible endoluminal prosthesis, it presents major limitations, such as those relating to providing the means for performing the expansion of the prosthesis in an aneurysm in the vicinity of a bifurcation (i.e. an abdominal aortic aneurysm) as well as preventing the control of fine adjustments required to any of the endoprosthesis components once implanted at the intended location. The delivery devices described in documents U.S. Pat. No. 6,673,102 (corresponding to CA 2503480) and U.S. Pat. No. 6,872,224 (corresponding to PI 9900959) allow greater flexibility for adjusting the different portions of the endoprostheses at their place of implant and use small diameter catheters but, nevertheless, present the same limitations as the system described in document U.S. Pat. No. 6,379,372.

Document U.S. Pat. No. 7,112,217 describes a delivery system and method for an endoprosthesis that allows adjustment of the various parts at the place of implant. However, this system and method presents the disadvantage that the link between the main body and the legs of the endoprosthesis is based on the coupling of stents fitted to the extremities of these parts and, furthermore, requires an incision of the artey to introduce the endoprosthesis implanting catheter.

Document US 2001/037142 reveals a delivery system and method for endovascular devices comprising: (i) a first sheath with distal and proximal extremities and at least a first expandable device at the proximal extremity, (ii) a second movable sheath inside the first sheath having respective distal and proximal extremities and containing a second expandable device and (iii) trigger buttons linked to the first and second expandable devices. Despite this system allowing the implant of na endoprosthesis and the adjustment of the various component parts, it neither provides the means of fine adjusting nor correcting the position of the endoprosthesis during the implant operation. The endoprosthesis delivery systems described in WO 01566504 (corresponding to documents US 2006/224227 and US 2003/220681) also present the same limitations.

Document US 2006/036314 describes a delivery system for endoprostheses that allows implanting the device in a bifurcated vessel but, however, this system does not allow any means of fine adjusting or correcting the position of the endoprosthesis during the implant operation.

Document US 2006/085012 illustrates a procedure for implanting na endoprosthesis using a delivery system without, however, describing implanting in a bifurcated vessel which is an operation requiring further steps for expanding the different parts of the endoprosthesis, such as, for example, the main body and the legs extending into the arteries branching from the trunk vessel in which the main body of the endoprosthesis is located. The delivery system described in document US 2006/142836 also presents similar failings. However, the delivery system described in US 2006/276872 (corresponding to PI 0414109) is intended for implanting this type of device in a curved vessel (i.e. the arched part of the aorta) where guide wire type delivery systems such as those described in documents WO 02051336 and WO 2005/039442 cannot be used. Despite the system described in US 2006/276872 being appropriate for curved sections of vessels such as the aorta, it does not meet the requirements for implanting endoprostheses in the vicinity of bifurcations and neither provides a means for correcting the position of the endoprosthesis during the implant process. O documento US 2006/085012

Although the endoprostheses and implanting systems mentioned above represent significant advances, mainly since they replace surgical techniques, it remains necessary to improve these devices as well as the methods for implanting them and applying them to varying biological conditions. Therefore, the present invention is directed at providing a safe and efficient means to treat aneurysms, including abdominal aortic aneurysms and thoracic aortic aneurysms thus eliminating a series of difficulties associated with current endoprostheses and their delivery systems. One of the aims of the endoprosthesis of the present invention is the prevention of endo-leakage disrupting the normal dynamic of vascular fluids by providing a device that is simple to position and replace, is an efficient fluid seal and, furthermore, has a configuratio that avoids any displacement at the place of implant without, however, interfering with the normal blood flow either in the actual vessel of the aneurysm or those vessels deriving from it. In summary, the device disclosed herein has the capacity of remaining firmly fixed and sealed tight when placed in bifurcated, tortuous, sharply curved or oddly shaped vessels, partially damaged or calcified vessels and either short or long vessels. Furthermore, the device of the present invention is extremely durable, extensible and may be reconfigured but, nevertheless, remains in position at the place of implant and maintains the sealing capacity required of an endoprosthesis.

### SUMMARY OF THE INVENTION

The invention intends to provide an endoprosthesis to be implanted in vessels damaged in a way as to be detrimental to the flow of the biological fluids they convey. More especially, the invention is intended for the treatment of aneurysms in areas of a vessel in the vicinity of a bifurcation, such as, for example, an aneurysm of the abdominal aorta.

A first embodiment provides an endoprosthesis comprising two complementary bodies, namely body “A” and body “B”, with the said bodies being formed by proximal, intermediate and distal regions and comprising a self-expanding structure of resistant and flexible material and a graft, wherein: (i) the said bodies “A” and “B” have a portion of greater diameter that divides, a bifurcating portion and at least two legs with one being significantly shorter than the other; (ii) the said proximal region of the said body “A” comprises a structure of resistant and flexible material, wherein its free end comprises a stent without a graft having a configuration appropriate for attachement to the vessel, wherein the remainder of the said body “A” comprising the said metal structure is stitched to the graft in at least two points; (iii) the said intermediate region of the said bodies “A” and “B” comprises a structure of resistant and flexible material stitched to the graft; (iv) the said distal region of the said bodies “A” and “B” has a free end without a graft comprising a configuration appropriate for attachement to the vessel walls, and; (v) the said bodies “A” and “B” are coupled in a manner that the said body “B” remains positioned within the said body “A” forming a sealed connection impeding leakage of the blood from inside the endoprosthesis to the aneurysm.

A second embodiment of the invention relates to a delivery system for an endoprosthesis inside a patient's vessel, wherein the said system comprises (a) proximal and distal supports disposed in a manner as to allow cooperative movement between themselves; (b) an external sheath intended to release the endoprosthesis in response to the relative and cooperative movement between the said proximal and distal supports; (c) proximal and distal cones that convey and fix the endoprosthesis in the intended position, and; (d) proximal and distal stent triggers connected to the said proximal and distal cones, respectively, wherein the said proximal and distal cones have independent movement in a manner as to allow correcting the precise location of the said endoprosthesis during the operation implanting it within the patient's vessel.

A third embodiment of the invention relates to the use of the delivery system for implanting an endoprosthesis in a patient needing such treatment wherein the system comprises (a) loading and fixing the invention endoprosthesis inside the delivery system by means of the proximal and distal cones; (b) positioning of the delivery system bearing the the invention endoprosthesis at the intended position within the vessel; (c) releasing the endoprosthesis from the external sheath by uncoupling the proximal support while maintaining the distal support unaltered; (d) correcting inadequate positioning of the endoprosthesis while the proximal and distal portions are attached to the proximal and distal cones, respectively; (e) uncoupling the distal support of the delivery system to position the free distal stent within the vessel, while maintaining the proximal support attached; (f) fine adjustment of the endoprosthesis position within the vessel to correct any remaining position errors; (g) release of the external sheath which results in the complete release of the endoprosthesis at the intended position, and; (h) removal of the delivery system from inside the patient.

A fourth embodiment of the invention relates to the use of the endoprosthesis in the treatment of aneurysms characterised by being in vessels conveying biological fluids having bifurcations.

### BRIEF DESCRIPTION OF THE DRAWINGS

FIG. 1 is an illustration of body “A” of the invention endoprosthesis.

FIG. 2 is an illustration of body “B” of the invention endoprosthesis.

FIG. 3 is an illustration of bodies “A” and “B” assembled, forming the invention endoprosthesis.

FIG. 4 illustrates a representation of the delivery system of the present invention.

FIG. 5 shows, in detail, the schematic procedure for the use of the delivery system of the present invention.

### DETAILED DESCRIPTION OF THE INVENTION

The endoprosthesis of the present invention is intended for the treatment of anomalies of the vessels conveying biological fluids (i.e. blood), more specifically in humans. More specifically, the invention endoprosthesis is percutaneous, which thus reduces or eliminates the surgical procedure normally necessary in cases of vessel deformation, such as, for example, blood vessels, with the said percutaneous endoprosthesis being preferably used in in the treatment of aneurysms, preferably of aneurysms occurring in the vicinity of bifurcations of the main vessel, more preferably of aortic aneurysms and, yet more preferably, abdominal aortic aneurysms. The fact that the device of the invention is percutaneous allows it to be implanted in the patient by mere puncture and, therefore, without needing to dissect any vessels (e.g. the iliac arteries) to allow access of the delivery system consisting of catheters that is required to position and release the implant (endoprosthesis). Without doubt, this constitutes a less aggressive system for the patient, avoiding the risks inherent to conventional treatment and thus reducing recovery time.

The endoprosthesis (E) of the present invention comprises an body “A” and an body “B”, wherein both these bodies are composed of a stable yet flexible metal structure, preferably a metal structure formed by a series of self-expanding stents and by the graft (R-CP and R-CCL) that cover most of the structure of stable material. Body “A” and body “B” are divided into three regions, namely; a proximal region, an intermediate region and a distal region.

The terms “proximal” and “distal” as used herein are intended to mean the portion closer to the heart and the portion furthest from the heart, respectively.

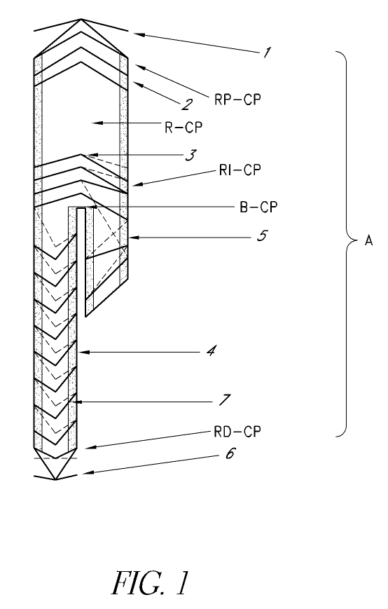

The proximal region of body “A” (RP-CP) presents a stent without a graft at its upper extremity (proximal) (1) or, therefore, graft free. Preferably, this free stent is of metal and more preferably it is made of nitinol wire which is a nickel-titanium alloy, having the main purpose of fixing the device to the walls of a vessel such as the aorta. As can be seen in FIG. 1, the free stent (1) has the shape of a suspended saddle that can be formed from one or more wires. In the case that the said suspended saddle is formed of several wires or, furthermore, of a wire having superposed regions, wherein the said wires are fitted with appropriate forms of attachment such as springs or some means of attachment by conformation and may be fitted with additional means of attachment, such as barbs or hooks that help fix the free stent (1) to the artery walls. Preferably, the free stent (1) is stitched to the graft at only two points, which contributes to reducing the transverse section of the endoprosthesis material (E).

It is important to note that the shape of the suspended saddle of the free stent (1) confers the endoprosthesis good resistance to possible displacement induced by the flow of biological fluids, i.e. blood, in the vessel. It should also be noted that endoprosthesis migration is a frequent and recurrent problem occurring with the devices presently available on the market.

With the intent of perfecting the attachment and adjustment of the endoprosthesis to the artery shape, the proximal region of the main body (RP-CP) can also be fitted with two or more saddle shaped wires stitched to the graft (R-CP) thus forming rings (2) that conform to the artery shape and make the endoprosthesis occupy the entire perimeter of the artery. Therefore, these rings (2) serve the purpose of seals for the proximal region and thus prevent blood flow from inside the endoprosthesis (E) to the artery walls. It should be noted that, preferably, a part of the main body does not have the metal structure allowing good adjustment of the endoprosthesis (E) in tortuous arteries.

It is also possible to note in FIG. 1 that the intermediate region of body “A” (RI-CP), preferably, presents a structure comprising a single shaped wire (3). This wire (3) has the purpose of maintaining the graft open and thus allowing unobstructed blood flow through the vessel. Optionally, this structure may also be reduced to a minimum leaving the contralateral body “B” to keep the endoprosthesis (E) open. The said intermediate region of the main body (RI-CP) contains a bifurcation (B-CP) from which two legs extend. One of these legs, leg (4), is longer and shall be positioned within one of the the main artery branches such as the iliac artery at the time of implant. The shorter leg, leg (5), has the purpose of serving as a seal between body “A” and body “B”.

FIG. 1 shows that the distal region of body “A” (RD-CP) also has a free stent (6) with the purpose of fixing the distal extremity of the endoprosthesis (E) to the walls of the vessel. Preferably, this free stent (6) is similar to free stent (1) having the shape of a suspended saddle and is stitched to the coating at only two points. Optionally, this region may be fitted with additional means of attachment, such as barbs or hooks that help fix the device. Preferably, the metal structure (7) of leg (4) is helicoidal in shape and is made of the same material used in the intermediate region of body “A” (RI_CP).

The materials used for the graft are natural, artificial or synthetic fibrous materials, coated or not, known in the art. The graft described in documents WO 2002/15951, PI 9608191 (corresponding to applications WO 9633066 and US 2005/096737) and WO 2005/025456 (corresponding to document CA 2539110) may be cited as examples. Preferably, the graft material is made of polyester (polyethylene terephthalate) or expanded polytetrafluorethylene (PTFE).

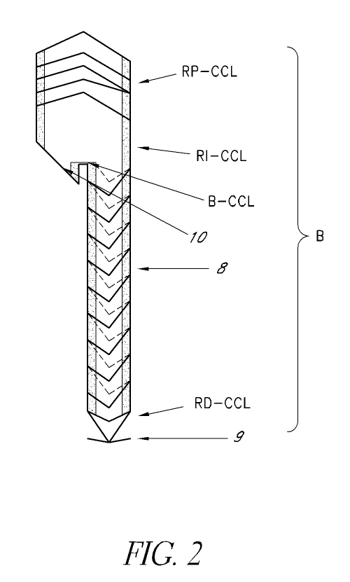

FIG. 2 shows body “B” of the endoprosthsis of the present invention. Body “B” is shorter than body “A” but also has a helical metal structure throughout its entire available transverse section that maintains the graft open. Optionally, the proximal region of body “B” (RP-CCL) may be provided with a free stent that may be similar to free stent (1) of the main body, or, alternatively, may be a conventional Z-stent (a Z shaped wire). The free stent of body “B” may be designed to occupy the portion of the main body without a metal structure thus increasing the rigidity and stability of the device when assembled.

The intermediate region of body “B” is provided with a bifurcation from which a contra-lateral leg extends, leg (8), that shall be positioned inside another branch of the main vessel (e.g. the iliac artery) when implanting the endoprosthesis (E). An opening (10) allowing blood flow is located to the side of the contra-lateral leg (9).

In this manner, both body “A” and body “B” are complementary and form a perfect bifurcation without any possibility of becoming disconnected. The walls of the main body are formed from a double layer of graft which twice thus confers twice the protection against the effects of the repeated secondary demands imposed by the pulsing of biological fluids (e.g. blood).

The distal region of body “B” (RD-CCL) is provided with a free stent (9) that, prefereably, is similar to free stent (6) of the leg (4) of the main body (body “A”), or, alternatively, may be a Z-stent.

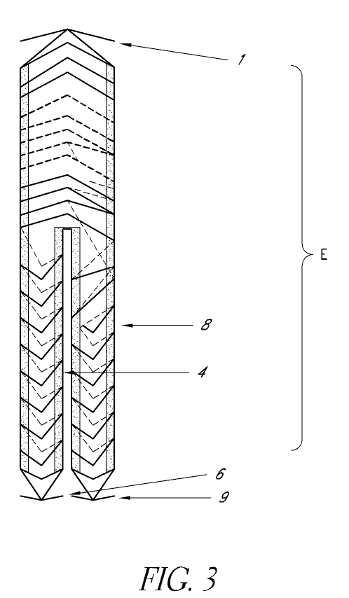

FIG. 3 shows the endoprosthesis (E) of the present invention entirely assembled. Bodies “A” and “B” are assembled by inserting body “B” inside body “A”. The opening (10) of body “B” is aligned with the longer leg (4) of body “A” and the longer leg (8) of body “B” passes through the short leg (5) of body “A”. This configuration allows free blood flow to both branches of the trunk vessel (e.g. the two iliac arteries). Furthermore, the superposition of the short leg (5) of body “A” and the long leg (8) of body “B” forms a seal between both bodies and prevents blood leakage.

Different from other devices currently available on the market that are based on the concept of two superposed endoprostheses (a bifurcated main body and a contra-lateral extension), the configuration of the endoprosthesis of the present invention confers the device good stability and completely eliminates any possibility of contra-lateral leg disconnection.

The division of the device structure into two bodies (“A” and “B”) constitutes a further advantage of the endoprosthesis of the present invention because it divides the material area and thus makes it possible to implant both bodies separately using small calibre catheters (e.g. a 14F catheter). Furthermore, the endoprosthesis of the present invention may be provided with sensory means for measuring and monitoring the patient's condition and position, such as, for example, the sensor device described in document WO 2004/105637

A second embodiment of the present invention relates to a delivery system for delivering the endoprosthesis of the present invention to the intended place of implant within a vessel conveying biological fluids (e.g. blood) in a patient.

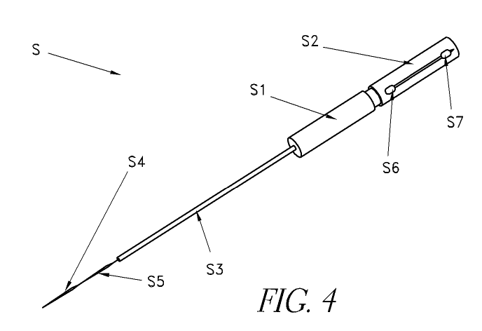

FIG. 4 shows the delivery system (S) for both bodies “A” and “B”, comprising a catheter with a distal and proximal endoprosthesis attachment device. This allows complete controle on the part of the surgeon when implanting the device. The delivery system (S) of the present invention comprises the following mechanisms for conveying, positioning, fine adjustment and attachment of the endoprosthesis (E) of the present invention: (a) proximal (S1) and distal supports (S2), (b) external sheath (S3), (c) proximal (S4) and distal cones (S5), (d) distal stent trigger (S6) and (e) proximal stent trigger (S7).

The proximal support (S1) is connected to the external sheath (S3) by the means of attachment of the latter, such as, for example, a flange. This configuration establishes a thread type connection between the proximal support (S1) and the distal support (S2) that allows the external sheath (S3) sufficient movement to release the endoprosthesis as a result of the relative movement of the proximal (S1) and distal supports (S2) (through a torsion movement such as that required for threaded connections).

In the delivery system (S) of the present invention, the proximal cone (S4) is connected to the proximal trigger (S7) by a tube, in the same mannner that the distal cone (S5) is connected to the distal trigger (S6). The distal (S6) and proximal triggers (S7) may be of any appropriate shape conducive to easy handling, such as, for example, sliding buttons that, when pressed with sufficient pressure to overcome their return spring tension, slide along serrated elements that fix them to the distal support (S2). In this manner, the distal (S6) and proximal triggers (S7) may slide both forwards and backwards, thus allowing sliding movement of the respective proximal (S4) and distal cones (S5) that are connected to the said distal (S6) and proximal triggers (S7) by means of tubes. Both cones are independent of each other and, therefore, depending on the movement of the respective triggers, may move independently of each other.

The delivery system (S) of the present invention has a simple positioning and release procedure for the endoprosthesis of the present invention as well as allowing the necessary correction and fine adjustment in the case of innaccurate positioning. The endoprosthesis (E) is released from the external sheath (S3) of the reduced diameter catheter (e.g. a 14F catheter) through a torsion mechanism (or the equivalent) between the proximal support (S1) and the distal support (S2) such as a thread type system. It should be stressed that the delivery system (S) of the present invention allows complete controle on the part of the surgeon when implanting the endoprosthesis of the present invention in a manner as to afford extreme precision.

The operation of the delivery system (S) of the present invention comprses the following consecutive steps: (i) loading and attaching the endoprosthesis (E) by means of the proximal (S4) and distal cones (S5), that are in turn connected to their respective proximal (S7) and distal (S6) free stent triggers. The endoprosthesis (E) is attached at both its proximal and distal extremities which allows any necessary correction in the case of innaccurate positioning during the release of the said endoprosthesis (E) or during the critical stages of the surgical procedures. A third embodiment of the invention relates to the use of the delivery system for implanting an endoprosthesis (E) in a patient requiring such treatment. The procedure for use may be described in the following manner:
the endoprosthesis (E) of the present invention is first loaded in the delivery system (S) of the present invention and connected to the proximal (S4) and distal cones (S5);
once the delivery system loaded with the endoprosthesis (E) is correctly positioned inside the artery, (e.g. the abdominal aorta), the surgeon starts the release procedure of the proximal support (S1) by holding the distal support (S2) with one of his hands while twisting the proximal support (S1) with the other hand (unscrewing the thread system between (S1) and (S2)). This operation releases the endoprosthesis (E) from the external sheath (S3);—the endoprosthesis (E) is released at this stage but, however, the proximal and distal parts remain connected to the proximal (S4) and distal cones (S5) thus allowing the surgeon to correct inaccurate positioning by moving the entire delivery system back or forward;
when the correct position has been encountered, the surgeon starts the procedure for releasing the distal support (S2) in the following manner: holding (S1) with one of his hands, the buttons (S6) are pressed and moved forward with the other hand. This operation releases the distal free stent (6) in the artery and only leaves the endoprosthesis (E) connected to the delivery system in the region of the proximal stent (1).
once the distal free stent (6) has been released, the surgeon may still correct any incorrect positioning (by fine adjustment) but, however, only by moving the entire system forward;
when the system is finally correctly positioned, the surgeon starts the procedure for releasing the external sheath (S3) by holding the proximal support (S1) with one of his hands while pressing and moving buttons (S7) forward with the other hand. The endoprosthesis (E) is now entirely released inside the artery and completely disconnected from the delivery system;
lastly, the delivery system (S) for the endoprosthesis may be retreived from the patient by withdrawing it.

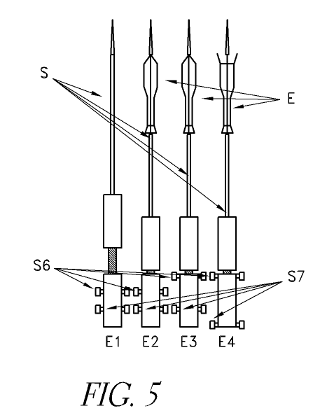

In summary, as can be seen from FIG. 5, the procedure for the use of the delivery system (S) of endoprosthesis (E) of the present invention may be performed according to the following stages:
in E1, endoprosthesis (E) is placed in position already loaded and connected;—in E2, (S1) is released through torsion of the proximal support (thread screwing movement);
in E3, (S2) is released through pressure and forward movement of buttons (S6) of the distal trigger; and
in E4, (S3) is released through pressure and backward movement of buttons (S7) of the proximal trigger.

All patent applications and publications mentioned in the above description are indicative of the level of expertise of those skilled in the art relating to the invention. All the patent applications and publications are included herein as reference in the same extent that each individual patent application or publication was specifically indicated to be indicated as reference.

Despite the above invention having had certain details described by means of illustrations and examples for the purpose of greater clarity and understanding, it remains obvious that certain alterations and modifications may be undertaken within the scope of the claims that accompany this description.

What is claimed is:

1. Modular endoprosthesis characterised by comprising; (a) a body “A” and (b) a body “B”, with the said bodies being formed by proximal, intermediate and distal regions and comprising a self-expanding structure of resistant and flexible material and a graft, wherein:
(i) the said bodies “A” and “B” have a portion of greater diameter that divides, in a bifurcating portion, in at least two legs with one being significantly shorter than the other;
(ii) the said proximal region of said body “A” comprises a structure of resistant and flexible material wherein its free end comprises a stent without graft having a configuration appropriate for attachment to the vessel wherein the remainder of the said body “A” comprising the said metal structure is stitched to the graft in at least two points;
(iii) the said intermediate region of the said bodies “A” and “B” comprises a structure of resistant and flexible material stitched to the graft;
(iv) the said distal region of the said bodies “A” and “B” has a free end without a graft comprising a configuration appropriate for attachment to the vessel walls, and;
(v) the said bodies “A” and “B” are coupled in a manner that the said body “B” remains positioned within the said body “A” forming a sealed connection impeding leakage of the blood from inside the endoprosthesis to the aneurysm and the disconnection of the modules (bodies “A” and “B”).

2. Endoprosthesis in accordance with claim 1, characterised by being a percutaneous endoprosthesis.

3. Endoprosthesis in accordance with claim 2, characterised by the fact that the percutaneous endoprosthesis is used in the treatment of aneurysms of the abdominal aorta.

4. Endoprosthesis in accordance with claim 1, characterised by the fact that the said metal structure of the proximal region of said body “A” is stitched to the graft at two points.

5. Endoprosthesis in accordance with claim 4, characterised by the fact that the said structure of resistant and flexible material is a metal structure.

6. Endoprosthesis in accordance with claim 5, characterised by the fact that the said metal structure is a structure of nickel-titanium alloy.

7. Endoprosthesis in accordance with claim 1, characterised by the fact that the said graft is made of polyethylene terephthalate.

8. Endoprosthesis in accordance with claim 1, characterised by the fact that the said appropriate shape of the free extremity without graft of the distal region of said bodies “A” and “B” is a suspended saddle.

9. Delivery system for the endoprosthesis of claims 1-7 or 8, characterised by the fact that the said system comprises:
(a) proximal and distal supports disposed so as to allow cooperative movement between themselves;
(b) an external sheath capable of releasing the endoprosthesis in response to the relative and cooperative movement between the said proximal and distal supports;
(c) proximal and distal cones that convey and attach the endoprosthesis in the intended position of implant, and;
(d) proximal and distal stent triggers connected to the said proximal end distal cones, respectively, wherein the said proximal and distal cones have independent movement in a manner as to allow correcting the location of the said endoprosthesis during the operation implanting it within the patient's vessel.

10. Use of the delivery system for implanting an endoprosthesis in a patient needing such treatment characterised by the fact that the system comprises:
(a) loading and connecting the endoprosthesis defined in claims 1-7 or 8 inside the delivery system defined in claim 9 by means of the proximal and distal cones;
(b) positioning of the delivery system bearing the invention endoprosthesis at the intended position within the vessel;
(c) releasing the endoprosthesis from the external sheath by uncoupling the proximal support while maintaining the distal support unaltered;
(d) correcting inadequate positioning of the endoprosthesis while the proximal and distal portions are connected to the proximal and distal cones, respectively;
(e) uncoupling the distal support of the delivery system to position the free distal stent within the vessel, while maintaining the proximal support attached;
(f) fine adjustment of the endoprosthesis position within the vessel to correct any remaining position errors;
(g) release of the external sheath which results in the complete release of the endoprosthesis at the intended position of implant, and;
(h) removal of the delivery system from inside the patient.

11. Use of the endoprosthesis defined in claims 1-7 or 8 in the treatment of aneurysms characterised by being in vessels conveying biological fluids having bifurcations.
